(19) United States
(12) Patent Application Publication
(10) Pub. No.: US 2013/0158624 A1
(43) Pub. Date: Jun. 20, 2013

(54) INCONTINENCE TREATMENT

(71) Applicants: Duncan Bain, Kings Langley (GB); Arthur Tudor Tucker, Leytonstone (GB)

(72) Inventors: Duncan Bain, Kings Langley (GB); Arthur Tudor Tucker, Leytonstone (GB)

(73) Assignee: SKY MEDICAL TECHNOLOGY LTD., Liverpool (GB)

(21) Appl. No.: 13/704,337

(22) PCT Filed: Jun. 13, 2011
(86) PCT No.: PCT/GB2011/051091

§ 371 Date: Feb. 22, 2013

(30) Foreign Application Priority Data
Jun. 15, 2010 (GB) 1009977.8

(51) Int. Cl. A61N 1/36

(52) U.S. Cl. CPC A61N 1/3606
USPC 607/41

(57) ABSTRACT

The present invention describes the use of transcutaneous electrical stimulation of a nerve emanating from the sacral plexus for the treatment or alleviation of incontinence. In preferred embodiments, the lateral popliteal nerve is stimulated in the region of the popliteal fossa. The stimulation is noninvasive.

### FIELD OF THE INVENTION

The present invention relates to a treatment for incontinence, particularly urinary incontinence, but also faecal incontinence.

### BACKGROUND TO THE INVENTION

Incontinence is a common and distressing problem, which involves involuntary leakage of urine and/or faecal matter. Urinary incontinence is more common in women than in men, and is often associated with a malfunction of the nerves which control bladder function—either the urethral sphincter may involuntarily relax, or the detrusor muscles which expel urine from the bladder may involuntarily contract, or both. Those nerves which control bladder function emanate from the lumbar and sacral segments of the spinal cord (the lumbrosacral plexus vertebral segments L5-S1), and in particular from the pudendal and coccygeal S1-S4 vertebral segments. The sciatic nerve is derived from the L4-S3 segments, and begins in the lower back and runs through the buttock and down the lower limb. The sciatic nerve branches into the tibial nerve and common peroneal nerve (also known as the common fibular, or popliteal nerve). The nerves controlling bladder function share commonality with the source of the sciatic nerve.

Treatment of incontinence may adopt medicated approaches, behavioural techniques, and/or electrical stimulation. Electrical stimulation to treat incontinence has been used for many years. The most common therapy involves the use of vaginal or rectal probes to deliver electrical current to the pelvic area, causing the pelvic floor muscles to contract, thereby improving function of these muscle groups (so called ‘tone’) and reducing the occurrence and severity of incontinence. However, the use of vaginal or rectal probes is obviously inconvenient for the patient and may be both painful and distressing.

An alternative form of electrical stimulation is direct stimulation of the relevant nerves. Two forms are known. Surgical implantation of a sacral nerve stimulator in the pelvis can provide continuous stimulation directly to the sacral area of the spinal cord, so reducing or preventing unwanted bladder contraction. However, this is an intrusive therapy, and surgical procedures have inherent risks. The other form of direct neural stimulation is known as Stoller Afferent Nerve Stimulation (SANS), and involves percutaneous stimulation of the posterior tibial nerve by means of a needle electrode inserted into the ankle of a user to directly stimulate the nerve. The tibial nerve is a mixed sensory-motor nerve comprised of the anterior rami of spinal roots L4 and S3. It comprises the outflow of the sacral nerves, which modulate the somatic and autonomic nervous supply to the pelvic floor, innervating directly the bladder, urinary sphincter, rectum and anal sphincter. As with sacral nerve stimulation, neurophysiological explanation of the effectiveness of percutaneous tibial nerve stimulation remains unclear. It is thought to work by a process called neuromodulation, whereby stimulation of the tibial nerve at the ankle results in an effect on the pelvic floor via action on the sacral nerves. The exact mechanisms behind neuromodulation, either central or peripheral, are yet to be explained. One theory suggests an improvement in blood flow to the pelvis whilst another possibility is a change in the neurochemical environment of neurons along the sacral pathways. Primate studies have shown that repetitive stimulation of the PTN exerts a strong inhibitory effect on nociceptive neurons of the spinothalamic tract. Some studies have suggested that there is an effect over and above placebo, but this also remains unclear.

Percutaneous tibial nerve stimulation serves to reduce or eliminate unwanted contractions of the bladder, so reducing incontinence. While the use of a needle electrode may be preferable to surgical implantation of a stimulation device, it is still invasive, and requires the patient to remain immobile in a sitting or supine position during use, thereby restricting the duration of individual treatment sessions. There is a need for an alternative form of electrical stimulation for treatment of incontinence.

Devices for transcutaneous electrical stimulation—that is, non-invasive stimulation—of nerves or muscles are known, typically for prevention or avoidance of deep vein thrombosis (DVT), or for reduction of pain (transcutaneous electrical nerve stimulation, TENS). The TENS protocol is also used for vaginal stimulation for treatment of incontinence.

One such device is that described in WO2006/054118, with further variations being described in PCT/GB2009/051713. These devices are intended to be used for stimulation of leg muscles via transcutaneous electrical stimulation. In brief, the devices include a pair of electrodes located on a support, which are placed on the skin of the user's leg, just behind the knee. The electrodes are activated, and repeated electrical impulses given to the user. The location of the electrodes on the user is such that the lateral and/or medial popliteal nerve is stimulated, causing contraction of the calf and foot muscles innervated by these nerves. As described in WO2006/054118, contraction of the calf and foot muscles in this way serves to activate the calf and foot musculovenous pumps which help to return blood to the heart and prevent pooling. This can be used to reduce the risks of DVT. An important feature of the method and device described in WO2006/054118 is that the stimulation may be used to activate opposed calf muscles, causing isometric contraction and little or no gross limb movement, and permits free mobilisation of the individual without restriction. This increases comfort for the user.

We have now surprisingly found that transcutaneous stimulation of the popliteal nerve can also be beneficial in treating or alleviating incontinence.

### SUMMARY OF THE INVENTION

According to a first aspect of the present invention, there is provided a method for treating and/or alleviating incontinence, the method comprising applying transcutaneous electrical stimulation to a limb of a patient such that a nerve emanating from the sacral plexus is stimulated. The sacral plexus emerges from the sacral vertebrae S1-S4, and bladder control is additionally governed by the sciatic nerve which branches from the sacral plexus. We have found that peripheral transcutaneous electrical stimulation of a nerve emanating from the sacral plexus may be sufficient to induce neural signals to travel along the nerve into the sacral plexus, and thence to the portion of the spine controlling bladder function, and/or bowel function. This can serve to treat or alleviate incontinence without the need for invasive neural stimulation such as provided by a needle or implantable device.

Preferably the nerve may be selected from the sciatic nerve, tibial nerve, and popliteal nerve, which all emanate from the sacral plexus. Preferably either or both of the lateral and medial popliteal nerves are stimulated. Most preferably the lateral popliteal nerve is stimulated in the region of the popliteal fossa; more specifically at the inner margin of the biceps femoris muscle, behind the fibula at the inner side of the tendon of the biceps femoris. It has not previously been known that non-invasive electrical stimulation in this specific area may be used in alleviating incontinence. Additionally or alternatively the medial popliteal nerve may be stimulated, which is located medially from the lateral popliteal nerve in the region of the popliteal fossa.

A typical electrical stimulus may be at a current of between 0 to 100 mA, preferably 0 to 50 mA, more preferably 1 to 40 mA, and most preferably between 1 to 20 mA. Preferably the electrical stimulus used is insufficient to elicit contraction of the muscles innervated by the relevant nerve; this reduces discomfort of the method to the user. However, in certain circumstances it may be that the stimulus necessary to effect treatment or alleviation of incontinence also causes muscular contraction; it is not a barrier to the use of the present method if muscular contraction is present, and indeed it is possible that muscular contraction may be contributory in effect in some individuals.

The stimulus may be an AC waveform, although it is preferably a DC waveform, more preferably a pulsed DC waveform. The stimulus may have a frequency of 0.01 to 100 Hz, preferably 0.1 to 80 Hz, more preferably 0.1 to 50 Hz; and most preferably 0.1 to 5 Hz. In other embodiments, the frequency may be from 30 to 60 Hz, and more preferably 40 to 50 Hz. Alternatively, a stimulus with a frequency from 0.1 to 1 Hz, or from 0.33 to 1 Hz may be used. The precise desired frequency may depend on the severity of the condition to be treated, and the general physical condition, age, sex, and weight of the patient, among other factors.

The stimulus may be applied for a duration between 0 and 1000 ms, between 100 and 900 ms, between 250 and 750 ms, between 350 and 650 ms, or between 450 and 550 ms. In certain embodiments, the stimulus may be applied for up to 5000 ms, up to 4000 ms, up to 3000 ms, or up to 2000 ms. Other durations may be used; again this may depend on the details of the patient.

Characteristics of the stimulus may vary over time. For example, a single stimulus may increase in current over the duration of the stimulus. Preferably the increase is gradual up to a peak; the stimulus may then either be maintained at the peak; terminate at the peak; or decrease in a gradual manner. Alternatively, where repeated stimuli are applied, characteristics of the stimuli may vary between different stimuli. For example, successive stimuli may be applied at increasing levels of current. Again, these successive stimuli may increase up to a peak gradually, followed by maintenance at that peak, or decrease from the peak. A cycle of increasing stimuli may be repeated a number of times.

In preferred embodiments, treatment is administered repeatedly over time. For example, a thirty minute stimulation treatment may be administered daily, or weekly. Treatment may be continued at intervals for days, weeks, months, or years. Where the stimulation used is insufficient to elicit muscular contraction, the patient may be able to undergo treatment for periods longer than thirty minutes at a time, or even largely continuously.

Preferably the treatment is for urinary incontinence, although it may also or instead be used for faecal incontinence due to the common and overlapping neurological pathway in the sacral plexus.

In preferred embodiments of the invention, the stimulation may be administered using a device as described in WO2006/054118, or as described in PCT/GB2009/051713. The reader is referred to those publications for further details of such devices. Of course, the present method is not restricted to use of those particular devices, any suitable device for administering transcutaneous electrical stimulation may be used.

According to a further aspect of the present invention, there is provided a device for treating or alleviating incontinence, the device comprising at least one transcutaneous electrode adapted to be located on a limb of a patient; a power supply connected to the electrode; and control means for activating the electrode such that transcutaneous electrical stimulation of a nerve emanating from the sacral plexus is effected; characterised in that the control means is adapted to activate the electrode so as to provide electrical stimulation sufficient to propagate a signal to the sacral plexus and thence to the nerves innervating the bladder and/or rectum. Preferably also the electrical stimulation is insufficient to cause muscle contraction of the muscles innervated by the stimulated nerve.

The electrode is preferably adapted to be located on the popliteal fossa of a patient.

The nerve to be stimulated is preferably selected from the sciatic nerve, tibial nerve, and popliteal nerve.

### BRIEF DESCRIPTION OF THE DRAWINGS

These and other aspects of the invention will now be described by way of example only and reference to the accompanying drawings, in which:

FIG. 1 shows an illustration of the placement of a transcutaneous stimulation device on the limb of a patient.

### DETAILED DESCRIPTION OF THE INVENTION

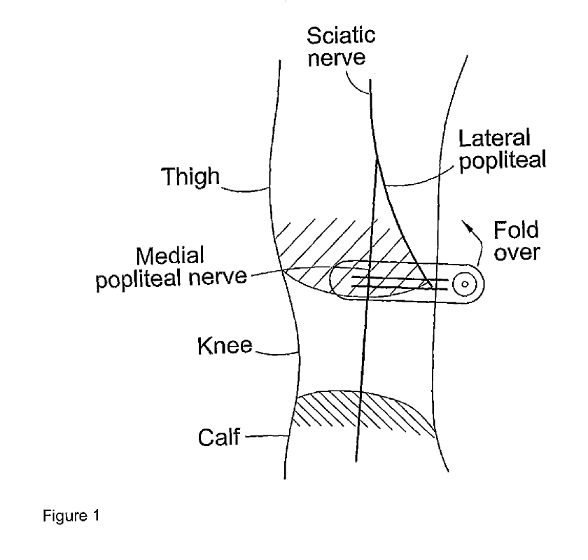

FIG. 1 shows a sketch of the posterior view of the right leg of a patient illustrating in general terms the location of the sciatic nerve, which descends from the sacral plexus, and which branches into the lateral and medial popliteal nerves. A transcutaneous stimulating device includes a pair of elongate electrodes coupled to a power source and control electronics, all mounted on a flexible elastomeric substrate. The electrodes are covered in a conductive gel, to promote electrical stimulation and to encourage adhesion of the device to the patient. The stimulating device is placed on the popliteal fossa of the patient, such that the elongate electrodes overlie the lateral and medial popliteal nerves. In other embodiments of the invention, the device may be placed to overlie only one of the lateral and medial popliteal nerves.

When the device is activated, the control electronics activate the electrodes to provide a 40 Hz pulsed DC of 20 mA for 0.1 second. This is repeated every 30 seconds for a period of 30 minutes. This forms one complete treatment cycle, which is given to the patient once weekly.

As the device is activated, the popliteal nerve is transcutaneously stimulated. Unlike the techniques described in, for example, WO2006/054118, in which the purpose of stimulation is to bring about muscle contraction of the limbs, the purpose of this stimulation is to cause propagation of a signal along the nerve to the sacral plexus, from where the signal stimulates the nerves serving the bladder. This is thought to condition the bladder to reduce or avoid excessive bladder contractions, so reducing the incidence of incontinence. The degree of stimulus used is insufficient to induce muscle contraction, so reducing discomfort for the user.

Evidence that percutaneous stimulation of the tibial nerve can serve to reduce incontinence has been published; for example:

Stoller, M. L. (1999). Afferent nerve stimulation for pelvic floor dysfunction. Eur Urol, 35 (suppl 2), 16;

Boyle, D. J., Prosser, K., Allison, M., Williams, N. S., & Chan, C. L. H. (2009). Percutaneous tibial nerve stimulation for the treatment of urge faecal incontinence. Colorectal Disease, I I (Suppl. 2), 2-8;

MacDiarmid, S. A., & Staskin, D. R. (2009). Percutaneous tibial nerve stimulation (PTNS): A literature-based assessment. Current Bladder Dysfunction Reports, 4, 29-33;

Shafik A, Ahmed I, EI-Sibai O, Mostafa R M. Percutaneous peripheral neuromodulation in the treatment of fecal incontinence. Eur Surg Res. 2003 March-April; 35(2):103-7;

Queralto M, Portier G, Cabarrot P H, Bonnaud G, Chotard J P, Nadrigny M, et al. Preliminary results of peripheral transcutaneous neuromodulation in the treatment of idiopathic fecal incontinence. Int J Colorectal Dis. 2006 October; 21(7):670-2;

de la Portilla F, Rada R, Vega J, Gonzalez C A, Cisneros N, Maldonado V H. Evaluation of the use of posterior tibial nerve stimulation for the treatment of fecal incontinence: preliminary results of a prospective study. Dis Colon Rectum. 2009 August; 52(8):1427-33;

Govaert B, Pares D, Delgado-Aros S, La Torre F, van Gemert W, Baeten C. A Prospective Multicenter Study to investigate Percutaneous Tibial Nerve Stimulation for the Treatment of Faecal Incontinence. Colorectal Dis. 2009 Aug. 5;

Chung J M, Lee K H, Hori Y, Endo K, Willis W D. Factors influencing peripheral nerve stimulation produced inhibition of primate spinothalamic tract cells. Pain. 1984 July; 19(3):277-93.

The present method differs from that described in these publications in that a different nerve and anatomical location is stimulated, and it is stimulated transcutaneously. However, we believe that this is sufficient to bring about the same or similar effects, so serving to treat incontinence. Evidence from previous trials for other conditions using transcutaneous stimulation of the popliteal nerve (described in WO2006/054118 and PCT/GB2009/051713) indicates that such stimulation is effective in eliciting neural signals in the popliteal nerve. As the popliteal nerve and the tibial nerve branch from the sacral plexus, we believe that this is powerful evidence that popliteal stimulation has a role to play in treatment of incontinence.

1. A method for treating and/or alleviating incontinence, the method comprising applying transcutaneous electrical stimulation to a limb of a patient such that a nerve emanating from the sacral plexus is stimulated.

2. The method of claim 1 wherein the nerve is selected from the sciatic nerve, tibial nerve, and popliteal nerve.

3. The method of claim 1 wherein either or both of the lateral and medial popliteal nerves are stimulated.

4. The method of claim 1 wherein the lateral popliteal nerve is stimulated in the region of the popliteal fossa.

5. The method of claim 1 wherein the electrical stimulus used is insufficient to elicit contraction of the muscles innervated by the relevant nerve.

6. The method of claim 1 wherein the stimulus is a pulsed DC waveform.

7. The method of claim 6 wherein the stimulus has a frequency of 0.1 to 100 Hz.

8. The method of claim 1 wherein treatment is administered repeatedly over time.

9. The method of claim 1 wherein treatment is continued at intervals for days, weeks, months, or years.

10. The method of claim 1 wherein treatment is administered in thirty minute blocks.

11. The method of claim 1 for treatment of urinary incontinence.

12. The method of claim 1 for treatment of faecal incontinence.

13. A device for treating or alleviating incontinence, the device comprising at least one transcutaneous electrode adapted to be located on a limb of a patient;
a power supply connected to the electrode; and control means for activating the electrode such that transcutaneous electrical stimulation of a nerve emanating from the sacral plexus is effected;
characterised in that the control means is adapted to activate the electrode so as to provide electrical stimulation sufficient to propagate a signal to the sacral plexus and thence to the nerves innervating the bladder and/or rectum.

14. The device of claim 13 wherein the control means is adapted to activate the electrode to provide an electrical stimulation which is insufficient to cause muscle contraction of the muscles innervated by the stimulated nerve.

15. The method of claim 7, wherein the frequency is from 30 to 60 Hz.

16. The method of claim 7, wherein the frequency is from 40 to 50 Hz.
